(19) United States
(12) Patent Application Publication
(10) Pub. No.: US 2025/0381371 A1
(43) Pub. Date: Dec. 18, 2025

(54) SHEATHED BAV DEVICE

(71) Applicant: BOSTON SCIENTIFIC SCIMED, INC., Maple Grove, MN (US)

(72) Inventors: Tim O'Connor, Galway (IE); Chris Cullen, Galway (IE); Pearse A Coffey, Galway (IE); Gavin O'Brien, Dublin (IE)

(73) Assignee: BOSTON SCIENTIFIC SCIMED, INC., MAPLE GROVE, MN (US)

(21) Appl. No.: 19/234,667

(22) Filed: Jun. 11, 2025

Related U.S. Application Data
(60) Provisional application No. 63/659,068, filed on Jun. 12, 2024.

(51) Int. Cl. A61M 25/10; A61M 25/00

(52) U.S. Cl. CPC A61M 25/10; A61M 25/0043; A61M 2025/0006; A61M 2025/006; A61M 2025/1081

(57) ABSTRACT

An outer sheath for delivering and re-sheathing an expandable balloon includes a tubular sheath having a distal end and a proximal end, and a plurality of balloon guide elements extending helically along an inner surface of a distal end region of the tubular sheath, where the plurality of balloon guide elements is spaced apart circumferentially. The outer sheath is part of a dilation balloon catheter assembly including a main catheter defining a lumen and a balloon catheter having a balloon fixed to a distal end thereof, where the balloon catheter is slidably disposed within the lumen of the main catheter, and the balloon is configured to move between a folded configuration and an expanded configuration. The plurality of balloon guide elements is configured to facilitate folding of the balloon after expansion when the balloon is pulled proximally into the outer sheath.

### CROSS-REFERENCE TO RELATED APPLICATION

This application is a continuation of U.S. Patent Application Ser. No. 63/659,068, filed Jun. 12, 2024, entitled “SHEATHED BAV DEVICE”, which is incorporated by reference herein in its entirety.

### TECHNICAL FIELD

The disclosure pertains to medical devices and more particularly to balloon aortic valvuloplasty (BAV) devices utilized in the transcatheter implantation of prosthetic stent-valves, and methods for using such medical devices.

### BACKGROUND

A wide variety of medical devices have been developed for medical use including, for example, medical devices involved in transcatheter aortic valve replacement (TAVR). Balloon catheters may be utilized to pre-dilate the aortic valve before implanting a prosthetic stent-valve. In some cases, a balloon may also be used post implant to ensure optimal expansion and anchoring of the prosthetic stent-valve. Of the known medical devices and methods, each has certain advantages and disadvantages. There is an ongoing need to provide alternative medical devices as well as alternative methods for manufacturing and using the medical devices.

### SUMMARY

This disclosure provides design, material, manufacturing method, and use alternatives for medical devices. An example outer sheath for delivering and re-sheathing an expandable balloon includes a tubular sheath having a distal end and a proximal end, and a plurality of balloon guide elements extending helically along an inner surface of a distal end region of the tubular sheath, the plurality of balloon guide elements being spaced apart circumferentially.

Alternatively, or additionally to the embodiment above, the plurality of balloon guide elements is a plurality of radially inwardly extending ridges.

Alternatively, or additionally to any of the embodiments above, a spacing between adjacent ridges is constant along an entire length of the ridges.

Alternatively, or additionally to any of the embodiments above, the tubular sheath has a total length of 100 mm to 150 mm and the plurality of ridges all have a length of 75 mm to 90 mm.

Alternatively, or additionally to any of the embodiments above, the tubular sheath has a wall thickness of 0.2 mm to 0.75 mm and the plurality of ridges all have a height of 0.2 mm to 0.75 mm.

Alternatively, or additionally to any of the embodiments above, the plurality of balloon guide elements includes three balloon guide elements.

Alternatively, or additionally to any of the embodiments above, the plurality of balloon guide elements includes six balloon guide elements.

Alternatively, or additionally to any of the embodiments above, the plurality of balloon guide elements includes nine balloon guide elements.

Alternatively, or additionally to any of the embodiments above, the plurality of balloon guide elements are grooves extending into the inner surface of the tubular sheath.

Alternatively, or additionally to any of the embodiments above, a proximal end region of the tubular sheath is devoid of any balloon guide elements.

Alternatively, or additionally to any of the embodiments above, the tubular sheath includes a radially outwardly extending disk fixed to an outer surface thereof at the proximal end.

An example dilation balloon catheter assembly includes a main catheter defining a lumen, a balloon catheter having a balloon fixed to a distal end thereof, the balloon catheter slidably disposed within the lumen of the main catheter, the balloon configured to move between a folded configuration and an expanded configuration, and an outer sheath slidably disposed over the main catheter, the outer sheath having a distal end and a proximal end and a plurality of balloon guide elements extending helically along an inner surface of a distal end region of the outer sheath, the plurality of balloon guide elements being spaced apart circumferentially and configured to facilitate folding of the balloon after expansion when the balloon is pulled proximally into the outer sheath.

Alternatively, or additionally to the embodiment above, the main catheter includes first and second hard stops spaced apart longitudinally at a proximal end region of the main catheter, wherein the outer sheath includes a disk disposed around an outer surface thereof, wherein the disk is disposed around the main catheter between the first and second hard stops and is slidable therebetween.

Alternatively, or additionally to any of the embodiments above, the first and second hard stops are proximal and distal protrusions fixed to and extending radially outward from an outer surface of the main catheter.

Alternatively, or additionally to any of the embodiments above, the plurality of balloon guide elements is a plurality of radially inwardly extending ridges.

Alternatively, or additionally to any of the embodiments above, a spacing between adjacent ridges is constant along an entire length of the ridges.

Alternatively, or additionally to any of the embodiments above, the outer sheath has a total length of 100 mm to 150 mm and the plurality of ridges all have a length of 75 mm to 90 mm.

Alternatively, or additionally to any of the embodiments above, the outer sheath has a wall thickness of 0.2 mm to 0.75 mm and the plurality of ridges all have a height of 0.2 mm to 0.75 mm.

Alternatively, or additionally to any of the embodiments above, the plurality of balloon guide elements are grooves extending into the inner surface of the outer sheath.

Another example dilation balloon catheter assembly includes a main catheter defining a lumen and having first and second hard stops spaced apart longitudinally at a proximal end region of the main catheter, a balloon catheter having a balloon fixed to a distal end thereof, the balloon catheter slidably disposed within the lumen of the main catheter, the balloon configured to move between a folded configuration and an expanded configuration, and an outer sheath slidably disposed over the main catheter, the outer sheath having a distal end and a proximal end and at least three radially inwardly extending ridges extending helically along an inner surface of a distal end region of the outer sheath, the ridges being spaced apart circumferentially and configured to facilitate folding of the balloon after expansion when the balloon is pulled proximally into the outer sheath, wherein the outer sheath includes a disk disposed around an outer surface thereof, wherein the disk is disposed around the main catheter between the first and second hard stops and is slidable therebetween.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 illustrates a balloon catheter extending distal of an example outer sheath;

FIG. 2 is a perspective view of an example outer sheath;

FIG. 3 is an end view of the outer sheath of FIG. 2;

FIG. 4 is a perspective view of another example outer sheath;

FIG. 5 is an end view of the outer sheath of FIG. 4;

FIG. 6A illustrates a deflated balloon distal of an example outer sheath;

FIG. 6B illustrates the balloon of FIG. 6A fully withdrawn into the outer sheath;

FIG. 7A illustrates a proximal end of an outer sheath over a BAV delivery catheter; and

FIG. 7B is a close-up view of the disk and hard stop control section of FIG. 7A.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

Additionally, the term “substantially” when used in reference to two dimensions being “substantially the same” shall generally refer to a difference of less than or equal to 5%. The terms “monolithic” and “unitary” shall generally refer to an element or elements made from or consisting of a single one-piece structure. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

Pre-dilatation of the native valve using an expandable balloon is often recommended for transcatheter aortic valve replacement (TAVR) procedures when using a self-expanding TAVR valve in order to prepare the anatomy for optimal valve implantation and to achieve a maximized post-implant effective orifice area. Pre-dilation may be performed using a balloon aortic valvuloplasty (BAV) balloon. After pre-dilation, it is desired for the balloon to be re-wrapped as much as possible to help with retraction of the balloon through the introducer sheath without damage to the balloon or the introducer sheath. Post dilatation rewrap is also important so the deflated balloon doesn't damage or move the implant. BAV balloons, currently used in TAVR procedures, are so large that they do not rewrap very well when pulled proximally into a conventional smooth-walled delivery sheath. The balloons tend to “pancake” or flatten out, creating an undesirably large cross-section.

In many cases there is a requirement for dilatation of the implant after it is implanted, in order to optimize expansion and anchoring of the implant and to eliminate paravalvular leak. Many physicians use the same BAV device both for pre and post-implantation dilation in order to save time and associated cost as well as to minimize the impact to patient safety.

To ensure maximum rewrap of the balloon and eliminate damage to both the balloon and the introducer sheath, an outer sheath as described in detail below may be added to the BAV device. The outer sheath may be advanced or retracted in an axial direction over the balloon to protect it during tracking, removal, and allow for redeployment. Advancing the outer sheath over the deflated balloon will force the balloon into a low profile so it can easily be retracted through an introducer sheath and associated hemostasis valve. The inner surface of the BAV outer sheath may be textured in order to encourage the deflated balloon wings to rewrap in the direction of the original heat set balloon folds as the balloon is pulled proximally into the outer sheath. Such texturing may include axially extending shallow tapered grooves or ridges on the inner surface of the outer sheath as described below.

The outer sheath will provide a slight increase of the BAV catheter profile, but this will still be significantly smaller than any TAVR device, and the outer sheath may provide additional shaft stiffness and improved push-ability which will prove beneficial in tortuous anatomies.

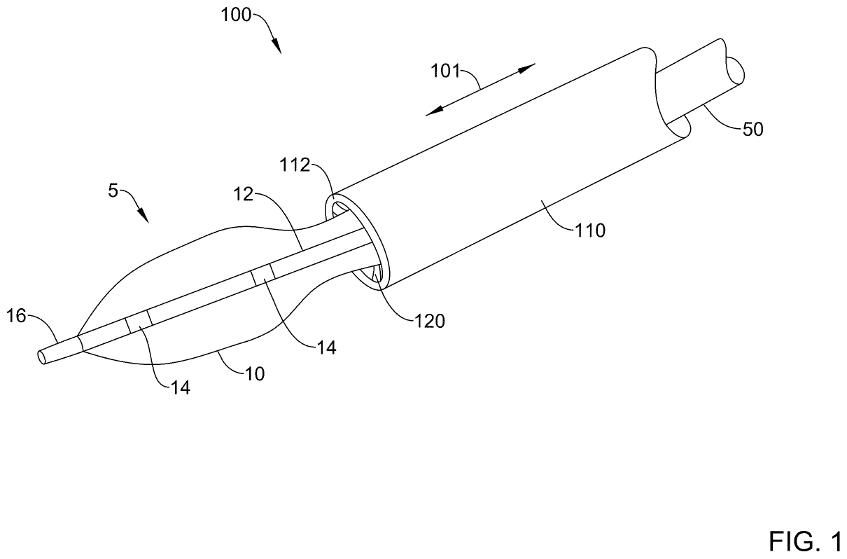

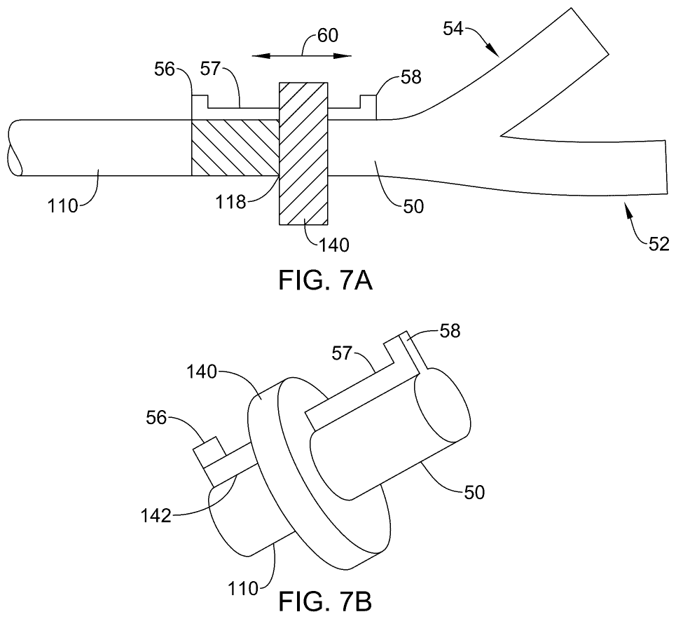

FIG. 1 illustrates an example dilation balloon catheter assembly 100, including a main catheter 50 defining a lumen, a balloon catheter 5 having an expandable balloon 10 fixed to a distal end region of a catheter shaft 12. The balloon catheter 5 is slidably disposed within the lumen of the main catheter 50, and the balloon 10 is configured to move between a folded configuration and an expanded configuration. The catheter shaft 12 may include one or more markers 14 disposed along the region surrounded by the balloon 10, and a distal tip 16 extending distally beyond the balloon 10. An outer sheath 110 may be slidable longitudinally over the main catheter 50, as shown by arrow 101. The outer sheath 110 may be configured to extend distally of a distal end of the main catheter 50 when the outer sheath 110 is in a distally advanced position. In this way, the outer sheath 110 may be advanced over the deflated balloon after dilation to aid in re-sheathing or refolding the balloon 10 before pulling the balloon catheter 5, folded within the distal end region of the outer sheath 110, proximally into the main catheter 50. The outer sheath 110 may have a distal end 112 and a proximal end 118 (see FIG. 7A), and a plurality of balloon guide elements 120 extending along an inner surface of at least a distal end region of the outer sheath.

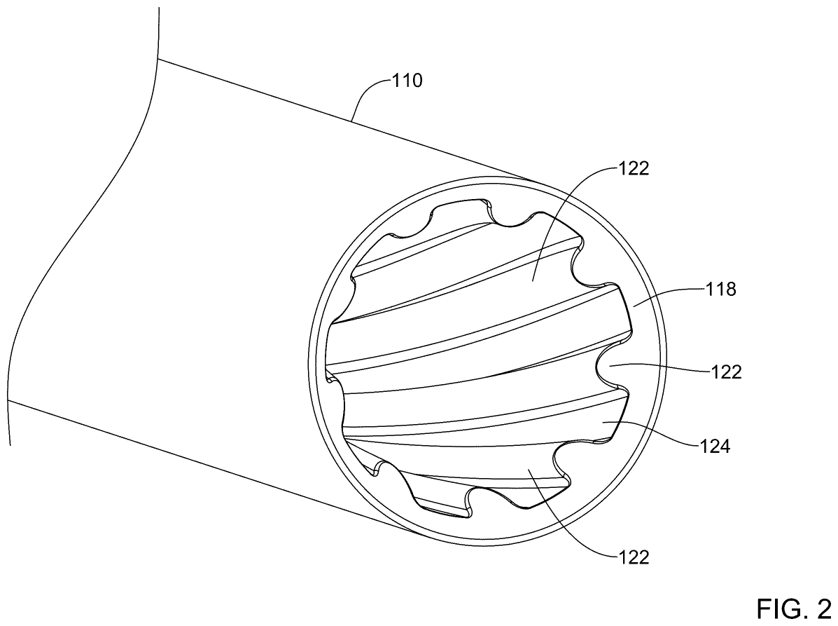

FIG. 2 illustrates one embodiment of an outer sheath 110 defined by a tubular sheath in which the plurality of balloon guide elements 120 are radially inwardly extending ridges 122 extending helically along the inner surface 124 of at least the distal end region of the outer sheath 110. The ridges 122 may be spaced apart circumferentially and extend to the distal end face 116 of the outer sheath 110. The spacing between adjacent ridges 122 may be constant along an entire length of the ridges.

The inner surface 124 of the outer sheath 110 between ridges 122 may be smooth. The number and spacing of ridges 122 may be selected for particular balloon sizes and/or materials in order to provide a desired folding configuration for the balloon. By advancing the outer sheath 110 over the balloon while the balloon catheter is rotated, the ridges 122 facilitate folding of the deflated balloon.

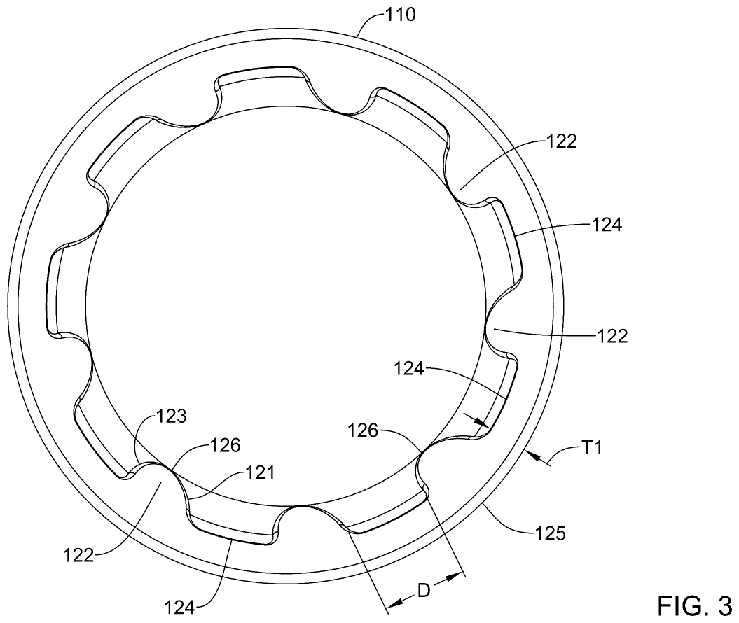

In some embodiments, the outer sheath 110 may have an outer diameter of 4.5 mm to 5.33 mm, and may have a total length of 100 mm to 150 mm. The plurality of ridges 122 may all have a length of 75 mm to 90 mm, leaving the proximal end region devoid of ridges along the inner surface. In other embodiments, the plurality of ridges 122 may extend continuously from the distal end to the proximal end of the outer sheath 110 along the entire length of the outer sheath. In the embodiment shown in FIGS. 2 and 3, the outer sheath 110 has nine ridges 122. In other embodiments, the outer sheath may have three ridges, six ridges, or any other number of ridges. In most embodiments, the outer sheath 110 will include at least three ridges 122. The distance D between ridges 122, whether there are three ridges or up to nine ridges, may be between 0.1 mm and 1.5 mm. The outer sheath 110 may have a wall thickness T1, measured between the inner surface 124 and the outer surface 125 of the outer sheath 110 of 0.2 mm to 1.00 mm, and the plurality of ridges may all have a height of 0.2 mm to 0.75 mm measured from the inner surface 124 of the outer sheath to an apex 126 of the ridge 122.

The plurality of ridges 122 may each have a first side 121 and a second side 123 that extend from the inner surface 124 and meet at the apex 126 of the ridge. In some embodiments, the first and second sides 121, 123 may extend radially inward from the inner surface 124 of the outer sheath 110 at different angles such that the ridge 122 leans in a first direction. In the embodiment shown in FIGS. 2 and 3, the first side 121 extends in an angle from the inner surface 124 of greater than 90° while the second side 123 extends in a substantially 90° angle from the inner surface 124. As a result, the ridges 122 lean to the left when viewed from the distal end of the outer sheath 110 (FIG. 3). In other embodiments, the ridges 122 may lean in the opposite direction, with the first side 121 extending in a substantially 90° angle from the inner surface 124, and the second side 123 extending in an angle from the inner surface 124 of greater than 90°. This leaning or angled orientation of the ridges 122 may facilitate in re-wrapping the balloon by catching or grabbing a portion of the deflated balloon and holding it while the balloon catheter is rotated, thereby re-folding the balloon.

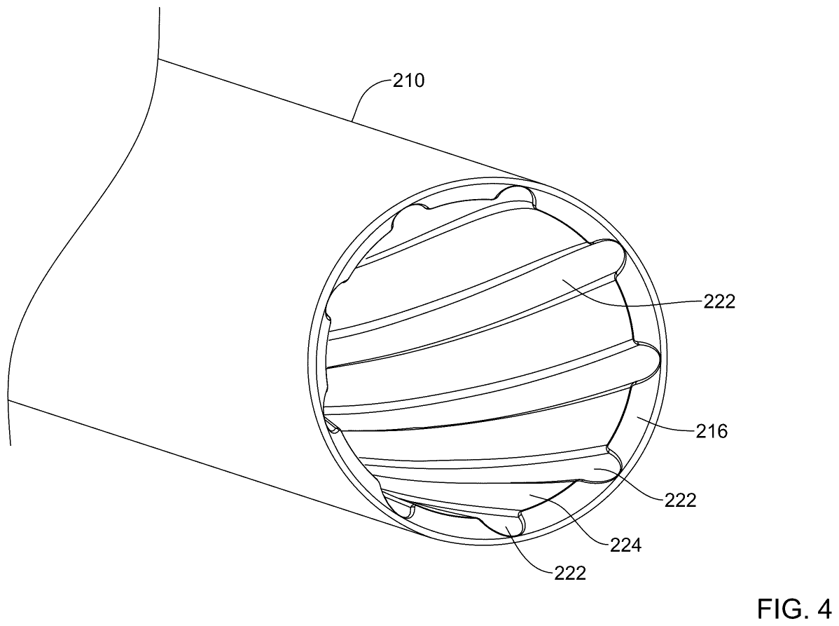

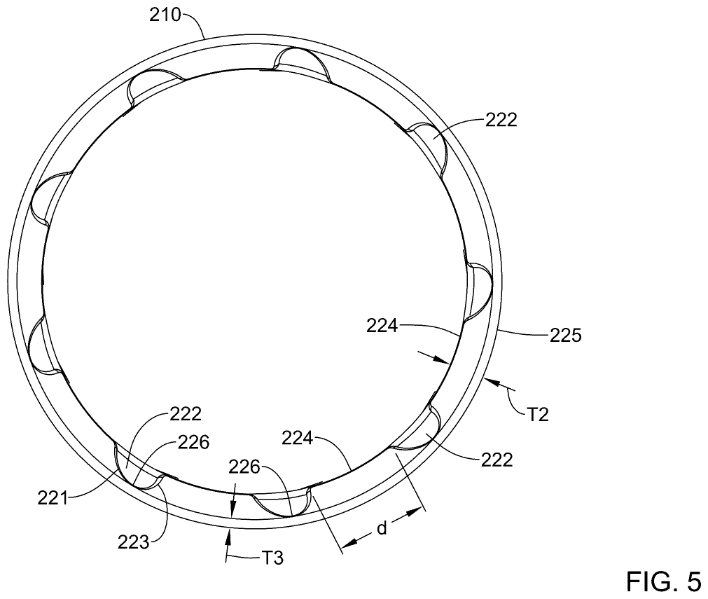

In another embodiment, the plurality of balloon guide elements 120 are grooves 222 extending radially into the inner surface 224 of the outer sheath 210, as shown in FIGS. 4 and 5. The plurality of grooves 222 may extend helically along the inner surface 224 of at least the distal end region of the outer sheath 210. The grooves 222 may be spaced apart circumferentially and extend to the distal end face 216 of the outer sheath 210. The spacing between adjacent grooves 222 may be constant along an entire length of the grooves.

The inner surface 224 of the outer sheath 210 between grooves 222 may be smooth, as shown in FIG. 4. The number and spacing of grooves 222 may be selected for particular balloon sizes and/or materials in order to provide a desired folding configuration for the balloon. By advancing the outer sheath 210 over the balloon while the balloon catheter is rotated, the grooves 222 facilitate folding of the balloon.

In some embodiments the outer sheath 210 may have a total length of 100 mm to 150 mm and the plurality of grooves 222 may all have a length of 75 mm to 90 mm, leaving the proximal end region devoid of grooves along the inner surface 224. In other embodiments, the plurality of grooves 222 may extend continuously from the distal end to the proximal end of the outer sheath 210. In the embodiment shown in FIGS. 4 and 5, the outer sheath 210 has nine grooves 222. In other embodiments, the outer sheath may have three grooves, six grooves, nine grooves, or any other number of grooves. In most embodiments, the outer sheath 210 will include at least three grooves 222. The distance d between grooves 222, whether there are three grooves or up to nine grooves, may be between 0.1 mm and 1.5 mm. The outer sheath 210 may have a wall thickness T2, measured between the inner surface 224 and the outer surface 225 of the outer sheath 210, of 0.2 mm to 1.00 mm. A minimum wall thickness T3 of 0.1 mm over the apex 226 of each groove 222 is necessary to retain the structural integrity and rigidity of the outer sheath 210 required for re-wrapping the balloon. When the wall thickness T2 of the outer sheath 210 is 0.2 mm, then the plurality of grooves may have a maximum depth of 0.1 mm, measured from the inner surface 224 of the outer sheath to an apex 226 of the groove 222. When the wall thickness T2 is 1.00 mm, the grooves 222 may have a maximum depth of 0.8 mm.

The plurality of grooves 222 may each have a first side 221 and a second side 223 that extend from the inner surface 224 and meet at the apex 226 of the groove. In some embodiments, the first and second sides 221, 223 may extend radially outward from the inner surface 224 of the outer sheath 210 at different angles such that the groove 222 leans in a first direction. In the embodiment shown in FIGS. 4 and 5, the first side 221 extends in an angle greater than 90° from the inner surface 224 while the second side 223 extends in a substantially 90° angle from the inner surface 224. As a result, the grooves 222 lean to the right when viewed from the end of the outer sheath 210 (FIG. 5). In other embodiments, the grooves may lean in the opposite direction, with the first side 221 extending in a substantially 90° angle from the inner surface 224, and the second side 223 extending in an angle greater than 90° from the inner surface 224. This leaning or angled orientation of the grooves 222 may facilitate in re-wrapping the balloon by catching or grabbing a portion of the deflated balloon and holding it while the balloon catheter is rotated, thereby re-folding the balloon.

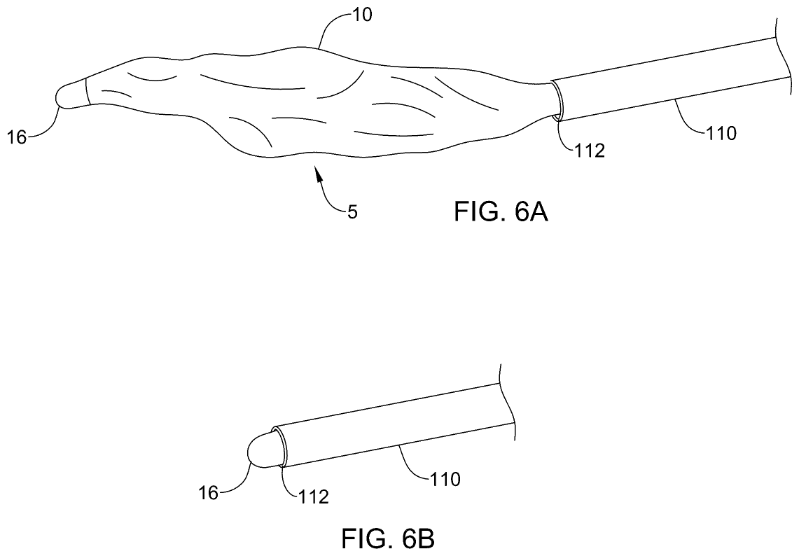

The following description of FIGS. 6A-7B refers to outer sheath 110, however it will be understood that the description applies equally to outer sheath 210. FIG. 6A shows a deflated balloon 10 just before being re-sheathed into the distal end 112 of the outer sheath 110. In some embodiments, the balloon catheter 5 may be configured to engage the distal end 112 of the outer sheath 110. For example, the distal tip 16 of the balloon catheter 5 may be sized to abut the distal end 112 of the outer sheath 110, making a smooth transition between the distal end of the balloon catheter and the outer sheath. As shown in FIG. 6B, once the outer sheath 110 has been advanced over the balloon 10, the distal tip 16 of the balloon catheter abuts and engages the distal end 112 of the outer sheath 110, defining a smooth transition therebetween, such that when the outer sheath 110 and balloon catheter are withdrawn through the main catheter of the BAV system, no part of the balloon catheter catches on any part of the system.

Proximal and distal movement of the outer sheath 110 over the main catheter 50 may be achieved via a radially outwardly extending disk 140 extending around and fixed to the outer surface of the proximal end 118 of the outer sheath 110. See FIG. 7A. The outer sheath 110 and disk 140 are disposed circumferentially around and slidable relative to the main catheter 50, with the disk 140 positioned between first and second hard stops 56, 58 fixed to the outer surface of the main catheter 50 adjacent the hub forming a guidewire lumen 52 and a balloon inflation lumen 54. The first and second hard stops 56, 58 may be projections extending radially outward from the main catheter 50, spaced apart longitudinally at the proximal end region of the main catheter 50. The distance between the first and second hard stops 56, 58 may correspond to the length of the balloon 10. In some embodiments, the first and second hard stops 56, 58 may be 30 mm to 100 mm apart. The first and second hard stops 56, 58 restrict longitudinal movement of the outer sheath 110 as the disk abuts each stop at opposite ends of sliding movement. The first and second hard stops may be proximal 58 and distal 56 protrusions fixed to and extending radially outward from the outer surface of the main catheter 50. In some embodiments, the first and second hard stops 56, 58 may be disposed on opposite ends of a bar or rod 57 fixed to the outer surface of the main catheter 50 adjacent its proximal end. The outer sheath 110 may have a longitudinal slit 142 configured to receive the first hard stop 56 and rod 57, if present. Manually moving the disk 140 back and forth between the first and second hard stops 56, 58, as shown by arrow 60 in FIG. 7A, causes the outer sheath 110 to move proximally off the balloon to deploy the balloon, when the disk is against hard stop 58, and to move distally over the balloon to re-sheath the balloon when the disk is against hard stop 56.

It will be understood that the dimensions described in association with the above figures are illustrative only, and that other dimensions of are contemplated. The materials that can be used for the various components of the outer sheath 110, 210 and dilation balloon catheter assembly 100, and the various elements thereof disclosed herein may include those commonly associated with medical devices. For simplicity purposes, the following discussion refers to the outer sheath 110 (and variations, systems or components disclosed herein). However, this is not intended to limit the devices and methods described herein, as the discussion may be applied to other elements, members, components, or devices disclosed herein.

In some embodiments, the outer sheath 110 and dilation balloon catheter assembly 100 (and all variations, systems or components thereof disclosed herein) may be made from a polymer or other suitable material generally used for medical catheters. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

In some embodiments, the disk 140, rod 57, and first and second hard stops 56, 58 may be made from a rigid polymer or other suitable material generally used for medical catheters. Some examples of suitable rigid polymers may include polycarbonate (PC), acrylonitrile butadiene styrene (ABS), polyether ether ketone (PEEK), high-density polyethylene (HDPE), ultra-high-molecular-weight-polyethylene (UHMWPE), polysulfone (PSU), and nylon.

In at least some embodiments, portions of the outer sheath 110 and dilation balloon catheter assembly 100 (and variations, systems or components thereof disclosed herein) may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the outer sheath 110 and dilation balloon catheter assembly 100 (and variations, systems or components thereof disclosed herein). Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the outer sheath 110 and dilation balloon catheter assembly 100 (and variations, systems or components thereof disclosed herein) to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An outer sheath for delivering and re-sheathing an expandable balloon, comprising:
a tubular sheath having a distal end and a proximal end; and
a plurality of balloon guide elements extending helically along an inner surface of a distal end region of the tubular sheath, the plurality of balloon guide elements being spaced apart circumferentially.

2. The outer sheath of claim 1, wherein the plurality of balloon guide elements is a plurality of radially inwardly extending ridges.

3. The outer sheath of claim 2, wherein a spacing between adjacent ridges is constant along an entire length of the ridges.

4. The outer sheath of claim 2, wherein the tubular sheath has a total length of 100 mm to 150 mm and the plurality of ridges all have a length of 75 mm to 90 mm.

5. The outer sheath of claim 2, wherein the tubular sheath has a wall thickness of 0.2 mm to 0.75 mm and the plurality of ridges all have a height of 0.2 mm to 0.75 mm.

6. The outer sheath of claim 1, wherein the plurality of balloon guide elements includes three balloon guide elements.

7. The outer sheath of claim 1, wherein the plurality of balloon guide elements includes six balloon guide elements.

8. The outer sheath of claim 1, wherein the plurality of balloon guide elements includes nine balloon guide elements.

9. The outer sheath of claim 1, wherein the plurality of balloon guide elements are grooves extending into the inner surface of the tubular sheath.

10. The outer sheath of claim 1, wherein a proximal end region of the tubular sheath is devoid of any balloon guide elements.

11. The outer sheath of claim 1, wherein the tubular sheath includes a radially outwardly extending disk fixed to an outer surface thereof at the proximal end.

12. A dilation balloon catheter assembly, comprising:
a main catheter defining a lumen;
a balloon catheter having a balloon fixed to a distal end thereof, the balloon catheter slidably disposed within the lumen of the main catheter, the balloon configured to move between a folded configuration and an expanded configuration; and
an outer sheath slidably disposed over the main catheter, the outer sheath having a distal end and a proximal end and a plurality of balloon guide elements extending helically along an inner surface of a distal end region of the outer sheath, the plurality of balloon guide elements being spaced apart circumferentially and configured to facilitate folding of the balloon after expansion when the balloon is pulled proximally into the outer sheath.

13. The dilation balloon catheter assembly of claim 12, wherein the main catheter includes first and second hard stops spaced apart longitudinally at a proximal end region of the main catheter, wherein the outer sheath includes a disk disposed around an outer surface thereof, wherein the disk is disposed around the main catheter between the first and second hard stops and is slidable therebetween.

14. The dilation balloon catheter assembly of claim 13, wherein the first and second hard stops are proximal and distal protrusions fixed to and extending radially outward from an outer surface of the main catheter.

15. The dilation balloon catheter assembly of claim 12, wherein the plurality of balloon guide elements is a plurality of radially inwardly extending ridges.

16. The dilation balloon catheter assembly of claim 15, wherein a spacing between adjacent ridges is constant along an entire length of the ridges.

17. The dilation balloon catheter assembly of claim 15, wherein the outer sheath has a total length of 100 mm to 150 mm and the plurality of ridges all have a length of 75 mm to 90 mm.

18. The dilation balloon catheter assembly of claim 15, wherein the outer sheath has a wall thickness of 0.2 mm to 0.75 mm and the plurality of ridges all have a height of 0.2 mm to 0.75 mm.

19. The dilation balloon catheter assembly of claim 14, wherein the plurality of balloon guide elements are grooves extending into the inner surface of the outer sheath.

20. A dilation balloon catheter assembly, comprising:
a main catheter defining a lumen and having first and second hard stops spaced apart longitudinally at a proximal end region of the main catheter;
a balloon catheter having a balloon fixed to a distal end thereof, the balloon catheter slidably disposed within the lumen of the main catheter, the balloon configured to move between a folded configuration and an expanded configuration; and
an outer sheath slidably disposed over the main catheter, the outer sheath having a distal end and a proximal end and at least three radially inwardly extending ridges extending helically along an inner surface of a distal end region of the outer sheath, the ridges being spaced apart circumferentially and configured to facilitate folding of the balloon after expansion when the balloon is pulled proximally into the outer sheath;
wherein the outer sheath includes a disk disposed around an outer surface thereof, wherein the disk is disposed around the main catheter between the first and second hard stops and is slidable therebetween.
